(19) United States
(12) Patent Application Publication
(10) Pub. No.: US 2020/0181236 A1
(43) Pub. Date: Jun. 11, 2020

(54) BISPECIFIC FUSION PROTEIN FOR IL-17 AND TNF-alpha

(71) Applicant: SHANGHAI KEXIN BIOTECH CO., LTD., Shanghai (CN)

(72) Inventors: Daocheng ZHU, Shanghai (CN); Jun BAO, Shanghai (US)

(21) Appl. No.: 16/637,246

(22) PCT Filed: Jan. 18, 2018
(86) PCT No.: PCT/CN2018/073271

§ 371 Date: Feb. 6, 2020

(30) Foreign Application Priority Data
Aug. 7, 2017 (CN) 201710668287.9

(51) Int. Cl. C07K 14/715

(52) U.S. Cl. CPC C07K 14/7151; A61K 38/00; C07K 14/7155

(57) ABSTRACT

The present invention belongs to the technical field of biology, and particularly relates to a bispecific fusion protein for IL-17 and TNF-α, and a preparation method and use thereof. The bispecific fusion protein for IL-17 and TNF-α of the present invention is a dimer, each includes three structural function areas, and the three structural function areas are a TNF-α receptor fragment, an Fcγ fragment and an IL-17 receptor fragment. The bispecific fusion protein provided by the present invention includes the IL-17 receptor fragment and the TNF-α receptor fragment, can effectively bind to IL-17 and/or TNF-α, respectively and has high bioactivity, specificity and stability.

### FIELD OF THE INVENTION

The present invention belongs to the technical field of biology, and particularly relates to a bispecific (dual-specific) fusion protein for IL-17 and TNF-α, and a preparation method and use thereof.

### BACKGROUND OF THE INVENTION

Cell factors in an interleukin-17 family are respectively named as interleukin-17A to interleukin-17F, and have their corresponding receptors respectively. The interleukin-17 cell factors may be bonded to corresponding receptor members to mediate different inflammatory reactions. The cell factors of the interleukin-17 family are just like a double-edged sword, which can be fast secreted to protect a body from being harmed by exogenous harmful substances in an acute inflammatory reaction, and can accelerate a disease course of various chronic diseases when a human body develops chronic inflammation due to various genetic and environmental factors. Therefore, the cell factors of the interleukin-17 family are closely related to human health, and a method of its adjusting and control mechanism also becomes a research hotspot in the art.

A tumor necrosis factor-α (TNF-α) is a cell factor capable of directly killing tumor cells, and has a corresponding receptor. The TNF-α is one of bioactive factors that have been found to have the highest direct tumor killing effect till now. In 1980s, clinical studies were performed in Europe and America, but were forced to end due to serious toxic and side effects. However, since the TNF-α has clear anti-tumor activity, scholars are reluctant to give up easily. In some clinic experiments, the TNF-α is used for isolated limb or organ perfusion, and sensational results are obtained. Meanwhile, through deep study on the TNF-α, some efficient low-toxicity TNF-α variants are developed, so that the important anti-tumor role of the TNF-α is confirmed again in Europe and America in the late 1990s.

There is no relevant report about the bispecific fusion protein of mutually binding interleukin-17 family and tumor necrosis factor-α (TNF-α).

### SUMMARY OF THE INVENTION

In view of defects in the prior art, the present invention is directed to provide a bispecific fusion protein for IL-17 and TNF-α, and a preparation method and use thereof for solving the problems in the prior art.

In order to achieve the above and other relevant objectives, according to a first aspect, the present invention provides a bispecific fusion protein for IL-17 and TNF-α. The bispecific fusion protein is a dimer. Each chain includes three structural function areas, and the three structural function areas are a TNF-α receptor fragment, an Fcγ fragment and an IL-17 receptor fragment.

The IL-17 receptor fragment is:

a) a polypeptide with an amino acid sequence shown as SEQ ID No. 1; or

b) a polypeptide with an amino acid sequence having 80% or more homology with the SEQ ID No. 1 and having functions of the polypeptide defined in a).

The TNF-α receptor fragment is:

c) a polypeptide with an amino acid sequence shown as SEQ ID No. 2; or

d) a polypeptide with an amino acid sequence having 80% or more homology with the SEQ ID No. 2 and having functions of the polypeptide defined in c).

The Fcγ fragment is an Fcγ Hinge-CH2-CH3 fragment, and the Fcγ Hinge-CH2-CH3 fragment is:

e) a polypeptide with an amino acid sequence shown as SEQ ID No. 3; or

f) a polypeptide with an amino acid sequence having 80% or more homology with the SEQ ID No. 3 and having functions of the polypeptide defined in e).

Specifically, the polypeptide in b) specifically refers to a polypeptide which is obtained from the polypeptide with an amino acid sequence shown as SEQ ID No. 1 through substitution, deletion or addition of one or more of (specifically, the number may be 1 to 50, may also be 1 to 30, may also be 1 to 20, may also be 1 to 10, may also be 1 to 5, or may also be 1 to 3) amino acids and has the functions of the polypeptide with an amino acid sequence shown as SEQ ID No. 1. The amino acid sequence of the polypeptide in b) may have 80% or more homology with the SEQ ID No. 1, may further specifically have 85% or more homology, may further specifically have 90% or more homology, may further specifically have 93% or more homology, may further specifically have 95% or more homology, may further specifically have 97% or more homology, and may further specifically have 99% or more homology.

Specifically, the protein sequentially includes the TNF-α receptor fragment, the Fcγ Hinge-CH2-CH3 fragment and the IL-17 receptor fragment from an end N to an end C, and is of a dimer structure.

Specifically, in the bispecific fusion protein for IL-17 and TNF-α provided by the present invention, the dimer is bonded through disulfide bonds between the Fcγ Hinge-CH2-CH3 fragments, so that an Fc fragment can be formed, a bimolecular TNF-α receptor fragment can be further formed at an end N of the Fc fragment, and a bimolecular IL-17 receptor fragment can be further formed at an end C of the Fc fragment.

Specifically, the bispecific fusion protein may be a bispecific fusion protein for IL-17 and/or TNF-α. The TNF-α receptor fragment may be a tumor necrosis factor receptor type II (TNFR2) fragment. The IL-17 receptor fragment may be an IL-17RA fragment.

According to a second aspect, the present invention provides an isolated polynucleotide, encoding the bispecific fusion protein for IL-17 and TNF-α.

Specifically, the isolated polynucleotide includes an IL-17 receptor fragment encoding sequence, an Fcγ fragment encoding sequence and a TNF-α receptor fragment encoding sequence.

Further specifically, the IL-17 receptor fragment encoding sequence is shown as SEQ ID No. 4, the TNF-α receptor fragment encoding sequence is shown as SEQ ID No. 5, and the Fcγ Hinge-CH2-CH3 fragment encoding sequence is shown as SEQ ID No. 6.

According to a third aspect, the present invention provides a recombinant expression vector which includes a polynucleotide for encoding the bispecific fusion protein for IL-17 and TNF-α.

Specifically, the recombinant expression vector is constructed by inserting the isolated polynucleotide into a multiple cloning site of an expression vector. The expression vector may be specifically an existing common expression vector well known to those in the art. The expression vector specifically includes but is not limited to a pET series expression vector, a pGEX series expression vector, a pcDNA series expression vector, etc. Those skilled in the art may select a proper vector, and may further modify the existing vector to construct and obtain a recombinant expression vector capable of reaching an expected expression level. The expected expression level may be a higher protein expression level, and may also be a relatively reasonable protein expression level so as to provide a reasonable dose for different individuals.

According to a fourth aspect, the present invention provides a fusion protein expression system including the recombinant expression vector or the polynucleotide in which an exogenous gene is integrated into a genome.

Specifically, the fusion protein expression system is constructed by transfecting the recombinant expression vector to a host cell. Any cell that is suitable for the expression vector to express can be used as the host cell, such as cells of yeast, insects, plants, etc. Preferably, the host cell is a eukaryotic cell, and may use a mammal animal host cell line incapable of generating an antibody. The cell line specifically includes but is not limited to: Chinese hamster ovary cells (CHO), baby hamster kidney cells (BHK, ATCC CCL 10), young rat sertoli cells, money kidney cells (COS cells), SV40 (COS-7, ATCC CRL 165 1) converted money kidney CVI cells, human embryo kidney cells (HEK-293), money kidney cells (CVI ATCC CCL 70), African green monkey kidney cells (VERO-76, ATCC CRL-1587), human cervical cancer cells (HELA, ATCC CCL 2), etc.

According to a fifth aspect, the present invention provides a preparation method of the bispecific fusion protein. The preparation method includes the following steps of:

1) culturing the fusion protein expression system so that the fusion protein expression system expresses the bispecific fusion protein;

2) collecting a culture material including the bispecific fusion protein; and

3) isolating the bispecific fusion protein from the culture material obtained in step 2.

Specifically, after a nucleotide sequence encoding the fusion protein of the present invention is obtained, a target fusion protein may be prepared and produced according to the following method. For example, a recombinant expression vector containing a polynucleotide encoding the target fusion protein is directly introduced into a host cell to obtain the fusion protein expression system, which is cultured under proper conditions, so that the expression of the encoded fusion protein is induced. The recombinant expression vector and the host cell used in the present invention are all those in the prior art, and can be directly commercially obtained. Culture media used in a culture process are various conventional culture media. Those skilled in the art may select applicable culture media according to experience for culture under the condition suitable for the growth of the host cell. After the host cell grows to a proper cell density, selected promoters are induced by a proper method (such as temperature conversion or chemical induction), and the cell is further cultured for a period of time. In the above-mentioned method, recombinant polypeptides may be expressed in a cell or on a cell membrane, mutually act to form a dimer fusion protein structure, and/or are secreted out of the cell. Once the bispecific fusion protein of the present invention is obtained, the bispecific fusion protein may be isolated and purified by various isolation methods based on its physical, chemical and other characteristics. Those methods are well known to those skilled in the art. Examples of those methods include but are not limited to: conventional renaturation treatment, treatment by a protein precipitation agent (a salting out method), centrifugation, permeation ultrasonication, super treatment, super centrifugation, molecular sieve chromatography (gel filtration), adsorption chromatography, ion exchange chromatography, high performance liquid chromatography (HPLC), various other liquid chromatography technologies, etc.

In one embodiment of the present invention, the bispecific fusion protein obtained through culture by the fusion protein expression system may be labeled by a method of inserting a marker in an expression vector, so as to facilitate the isolation and purification of the bispecific fusion protein in the culture material. The marker specifically may be various conventional markers applicable to the purification of fusion protein in the art.

According to a sixth aspect, the present invention provides a composition, including a therapeutically effective amount of culture material of the bispecific fusion protein or the fusion protein expression system (for example, host cells).

Herein, the reduction of one or more of symptoms or clinical indexes indicates that the therapy is effective.

Specifically, the composition further includes a pharmaceutically acceptable carrier. The pharmaceutically acceptable carrier refers to a carrier for therapeutic agent administration, includes various excipients and diluents, and specifically refers to such drug carriers which are not necessary active ingredients themselves and do not have excessive toxicity after use. Proper carriers are well known to those skilled in the art. There is full discussion about pharmaceutically acceptable excipients in Remington's Pharmaceutical Sciences (Mack Pub. Co., N.J. 1991). The pharmaceutically acceptable carrier in the composition may include liquid such as water, saline, glycerol and ethyl alcohol. Additionally, auxiliary substances, such as disintegrants, wetting agents, emulsifying agents, pH buffer substances, seaweed gel, pectin, sodium carboxymethylcellulose (CMC), xanthan gum, gellan gum, guar gum, carrageenan, sucrose, maltitol and stevioside may also exist in the carriers.

According to a seventh aspect, the present invention provides a use of the bispecific fusion protein in preparation or screening of a TNFα inhibitor and/or an IL-17 inhibitor.

The use specifically refers to: by using IL-17 and/or TNFα as an action target, the bispecific fusion protein as an active ingredient is used for preparing a therapeutic drug, the therapeutic drug may use modes of decreasing an expression amount of the IL-17 and/or the TNFα, inhibiting the activity of the IL-17 and/or the TNFα, etc. Specifically, decreasing an expression amount of the IL-17 specifically refers to that the expression amount of the IL-17 may be reduced by at least 10%, may be further specifically reduced by at least 30%, may be further specifically reduced by at least 50%, may be further specifically reduced by at least 70%, and may be further specifically reduced by at least 90% compared with that before administration. Specifically, decreasing an expression amount of the TNFα specifically refers to that the expression amount of the TNFα may be reduced by at least 10%, may be further specifically reduced by at least 30%, may be further specifically reduced by at least 50%, may be further specifically reduced by at least 70%, and may be further specifically reduced by at least 90% compared with that before administration. Specifically, inhibiting the activity of the IL-17 refers to that the activity of the IL-17 may be reduced by at least 10%, may be further specifically reduced by at least 30%, may be further specifically reduced by at least 50%, may be further specifically reduced by at least 70%, and may be further specifically reduced by at least 90% compared with that before administration. Specifically, inhibiting the activity of the TNFα refers to that the activity of the TNFα may be reduced by at least 10%, may be further specifically reduced by at least 30%, may be further specifically reduced by at least 50%, may be further specifically reduced by at least 70%, and may be further specifically reduced by at least 90% compared with that before administration.

Further specifically, the IL-17 may be IL-17A.

According to an eighth aspect, the present invention provides a therapeutic method of applying the drug composition to an individual.

The individual refer to an animal (including human) capable of accepting the drug composition and/or the therapeutic method, including both male and female genders herein, unless otherwise specified. Therefore, the individual at least includes any mammals, including but not limited to human, non-human primates such as mammals, dogs, cats, horses, goats, pigs, cows, etc., which can be benefited from the therapy by using the drug composition.

Specifically, the therapeutic method uses the modes of decreasing the expression amount of the IL-17 and/or the TNFα, inhibiting the activity of the IL-17 and/or the TNFα, etc.

Based on the above, the bispecific fusion protein provided by the present invention includes the IL-17 receptor fragment and the TNF-α receptor fragment, can effectively target the IL-17 and/or the TNF-α, and has high bioactivity, specificity and stability. The structure of the bispecific fusion protein can effectively reduce the dose and therapy cost, and the bispecific fusion protein has a very high industrialization value.

### BRIEF DESCRIPTION OF THE DRAWINGS

FIG. 1 shows a schematic diagram of a bispecific fusion protein for IL-17 and TNF-α provided by the present invention.

FIG. 2 shows an identification schematic diagram of a bispecific fusion protein for IL-17 and TNF-α provided by the present invention.

FIG. 3 shows an identification schematic diagram of a fusion protein for a TNF provided by the present invention.

FIG. 4 shows a schematic diagram of an activity experiment effect of a bispecific fusion protein for IL-17 and TNF-α provided by the present invention.

FIG. 5 shows a schematic diagram of a TIF bonding to TNF-α and IL-17, respectively of the present invention.

FIG. 6 shows a schematic diagram of TIF inhibiting toxicity effect of TNF-α on L929 cells of the present invention.

FIG. 7 shows a schematic diagram of inhibition on GRO-α generation in TNF-α and IL-17A induced HT-29 cells by TIF of the bispecific fusion protein for IL-17 and TNF-α provided by the present invention.

FIG. 8 shows a schematic diagram of TIF inhibiting generation of IL-17A and TNF induced CXCL1 in DBA/1 mice.

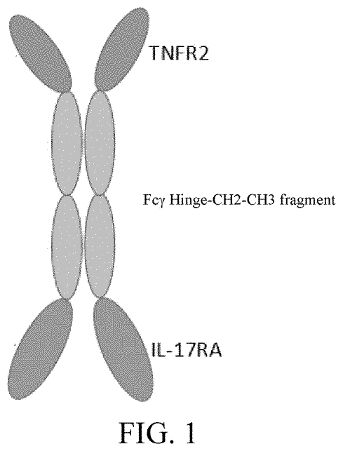

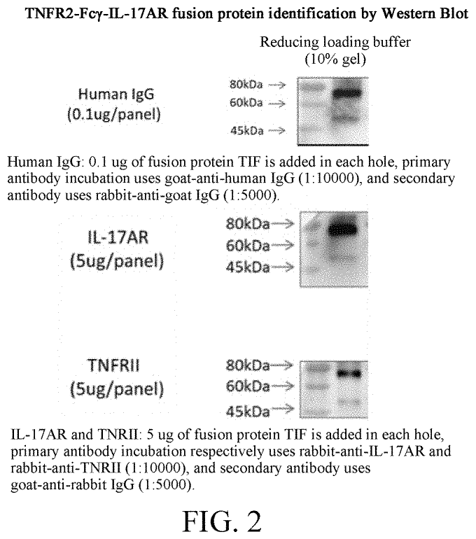

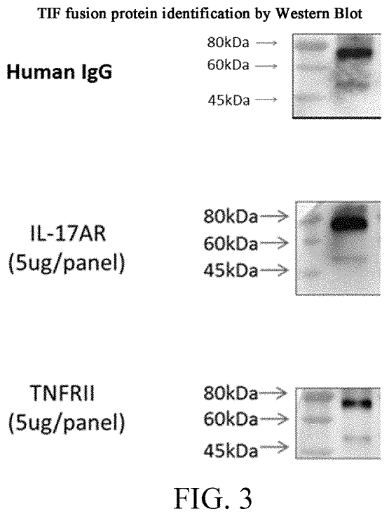

### DETAILED DESCRIPTION OF THE INVENTION

The implementations of the present invention will be described below with reference to specific examples. Those skilled in the art may easily understand other advantages and effects of the present invention by the contents disclosed in the present specification. The present invention may also be implemented or applied through other different specific implementations. Various modifications or changes may also be made on the details in the present specification without departing from the spirit of the present invention based on different viewpoints and applications.

Before the implementations according to the present invention is further described, it should be understood that the protection scope of the present invention is not limited to the specific implementations described below. It should also be understood that the term in the embodiments according to the present invention is used to describe the particular implementations, and is not intended to limit the protection scope of the present invention. In the specification and claims according to the present invention, unless otherwise stated specifically, the singular forms “a”, “an”, and “the” include the plural forms.

When the numerical ranges are given by the embodiments, it should be understood that the two endpoints of each numerical range and any numerical value between the two endpoints can be selected, unless otherwise stated herein. Unless otherwise defined, all technical and scientific terms used in the present invention have the same meaning as commonly understood by those skilled in the art. In addition to the specific methods, devices and materials, any methods, devices, and materials of the prior art that are similar or equivalent to the methods, devices, and materials described in the embodiment according to the present invention can also be used to implement the present invention in accordance with the prior art known by those skilled in the art and the description of the present invention.

Unless otherwise stated, the experimental methods, detection methods, and preparation methods disclosed in the present invention employ molecular biology, biochemistry, chromatin structure and analysis, analytical chemistry, cell culture, recombinant DNA technology conventional in the field, and conventional technology in related fields. These techniques have been well described in the existing literature, see Sambrook et al., MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, Chromatin (P. M. Wassarman and A. P. Wolffe, eds.), Academic Press, San Diego, 1999; and METHODS IN MOLECULAR BIOLOGY, Vol. 119, Chromatin Protocols (P. B. Becker, ed.) Humana Press, Totowa, 1999 and the like for details.

### Embodiment 1

1. An amino acid sequence of a TNFR2 fragment as a TNF-α receptor fragment is shown as SEQ ID No. 2, and an encoding sequence is shown as SEQ ID No. 5. An amino acid sequence of a Fcγ Hinge-CH2-CH3 fragment is shown as SEQ ID No. 3, and an encoding sequence is shown as SEQ ID No. 6. An amino acid sequence of an IL-17RA fragment as an IL-17 receptor fragment is shown as SEQ ID No. 1, and an encoding sequence is shown as SEQ ID No. 4.

The amino acid sequence of the TNFR2 fragment (SEQ ID No. 2) is:

Mapvavwaalavglelwaaahalpaqvaftpyapepgstcrlreyydqta
qmccskcspgqhakvfctktsdtvcdscedstytqlwmwvpeclscgsrc
ssdqvetqactreqnrictcrpgwycalskqegcrlcaplrkcrpgfgva
rpgtetsdvvckpcapgtfsnttsstdicrphqicnvvaipgnasmdavc
tstsptrsmapgavhlpqpvstrsqhtqptpepstapstsfllpmgpspp
aegstgd

The encoding sequence of the TNFR2 fragment (SEQ ID No. 5) is:

Atggcgcccgtcgccgtctgggccgcgctggccgtcggactggagctctg
ggctgcggcgcacgccttgcccgcccaggtggcatttacaccctacgccc
cggagcccgggagcacatgccggctcagagaatactatgaccagacagct
cagatgtgctgcagcaaatgctcgccgggccaacatgcaaaagtcttctg
taccaagacctcggacaccgtgtgtgactcctgtgaggacagcacataca
cccagctctggaactgggttcccgagtgcttgagctgtggctcccgctgt
agctctgaccaggtggaaactcaagcctgcactcgggaacagaaccgcta
ctgcacctgcaggcccggctggtactgcgcgctgagcaagcaggaggggt
gccggctgtgcgcgccgctgcgcaagtgccgcccgggcttcggcgtggcc
agaccaggaactgaaacatcagacgtggtgtgcaagccctgtgccccggg
gacgttctccaacacgacttcatccacggatatttgcaggccccaccaga
tctgtaacgtggtggccatccctgggaatgcaagcatggatgcagtctgc
acgtccacgtcccccacccggagtatggccccaggggcagtacacttacc
ccagccagtgtccacacgatcccaacacacgcagccaactccagaaccca
gcactgctccaagcacctccttcctgctcccaatgggccccagcccccca
gctgaagggagcactggcgac

The amino acid sequence of the Fcγ Hinge-CH2-CH3 fragment (SEQ ID No. 3) is:

Epkscdkthtcppcpapellggpsvflfppkpkdtlmisrtpevtcvvvd
vshedpevkfnwyvdgvevhnaktkpreeqynstyrvvsvltvlhqdwln
gkeykckvsnkalpapiektiskakgqprepqvytlppsreemtknqvsl
tclvkgfypsdiavewesngqpennykttppvldsdgsfflyskltvdks
rwqqgnvfscsvmhealhnhytqkslslspgk

The encoding sequence of the Fcγ Hinge-CH2-CH3 fragment (SEQ ID No. 6) is:

GAGCCCAAATCTTGTGACAAAACTCACACATGCCCACCGTGCCCAGCACC
TGAACTCCTGGGGGGACCGTCAGTCTTCCTCTTCCCCCCAAAACCCAAGG
ACACCCTCATGATCTCCCGGACCCCTGAGGTCACATGCGTGGTGGTGGAC
GTGAGCCACGAAGACCCTGAGGTCAAGTTCAACTGGTACGTGGACGGCGT
GGAGGTGCATAATGCCAAGACAAAGCCGCGGGAGGAGCAGTACAACAGCA
CGTACCGTGTGGTCAGCGTCCTCACCGTCCTGCACCAGGACTGGCTGAAT
GGCAAGGAGTACAAGTGCAAGGTCTCCAACAAAGCCCTCCCAGCCCCCAT
CGAGAAAACCATCTCCAAAGCCAAAGGGCAGCCCCGAGAACCACAGGTGT
ACACCCTGCCCCCATCCCGGGAGGAGATGACCAAGAACCAGGTCAGCCTG
ACCTGCCTGGTCAAAGGCTTCTATCCCAGCGACATCGCCGTGGAGTGGGA
GAGCAATGGGCAGCCGGAGAACAACTACAAGACCACGCCTCCCGTGCTGG
ACTCCGACGGCTCCTTCTTCCTCTATAGCAAGCTCACCGTGGACAAGAGC
AGGTGGCAGCAGGGGAACGTCTTCTCATGCTCCGTGATGCATGAGGCTCT
GCACAACCACTACACGCAGAAGAGCCTCTCCCTGTCCCCGGGTAAA

The amino acid sequence of the IL-17RA fragment (SEQ ID No. 1) is:

LRLLDHRAPVCSQPGLNCTVKNSTCLDDSWIHPRNLTPSSPKDLQIQLHF
AHTQQGDLFPVAHIEWTLQTDASILYLEGAELSVLQLNTNERLCVRFEFL
SKLRHHHKRWRFTFSHFVVDPGQEYEVTVHHLPKPIPDGDPNHQSKNFLV
PDCEDARMKVTTPCMSSGSLWDPNITVETLEAHQLRVSFTLWNESTHYQI
LLTSFPHMENHSCFEHMHHIPAPRPEEFHQRSNVTLTLRNLKWCCRHQVQ
IQPFFSSCLNDCLRHSVTVSCPEMPDTPEPIPDYMPLW

The encoding sequence of the IL-17RA fragment (SEQ ID No. 4) is:

Ctgcgactcctggaccaccgggcgccagtctgctcccagccggggctaaa
ctgcacggtcaagaatagtacctgcctggatgacagctggattcaccctc
gaaacctgaccccctcctccccaaaggacctgcagatccagctgcacttt
gcccacacccaacaaggagacctgttccccgtggctcacatcgaatggac
actgcagacagacgccagcatcctgtacctcgagggtgcagagttatctg
tcctgcagctgaacaccaatgaacgtttgtgcgtcaggtttgagtttctg
tccaaactgaggcatcaccacaaacggtggcgttttaccttcagccactt
tgtggttgaccctggccaggaatatgaggtgaccgttcaccacctgccca
agcccatccctgatggggacccaaaccaccagtccaagaatttccttgtg
cctgactgtgaggacgccaggatgaaggtaaccacgccatgcatgagctc
aggcagcctgtgggaccccaacatcaccgtggagaccctggaggcccacc
agctgcgtgtgagcttcaccctgtggaacgaatctacccattaccagatc
ctgctgaccagttttccgcacatggagaaccacagttgctttgagcacat
gcaccacatacctgcgcccagaccagaagagttccaccagcgatccaacg
tcacactcactctacgcaaccttaaatggtgctgtcgccaccaagtgcag
atccagcccttcttcagcagctgcctcaatgactgcctcagacactccgt
gactgtttcctgcccagaaatgccagacactccagaaccaattccggact
acatgcccctgtgg

2. A fusion protein TNFR2-Fcγ-IL-17RA-Fc sequentially includes a TNFR fragment, an Fcγ Hinge-CH2-CH3 fragment and an IL-17RA fragment from an end N to an end C.

3. An encoding sequence of the fusion protein TNFR2-Fcγ-IL-17RA-Fc sequentially includes a TNFR fragment encoding sequence, an FcγHinge-CH2-CH3 fragment encoding sequence and an IL-17RA fragment encoding sequence from an end N to an end C.

An amino acid sequence of the TNFR2-Fcγ-IL-17RA-Fc is:

MAPVAVWAALAVGLELWAAAHALPAQVAFTPYAPEPGSTCRLREYYDQTA
QMCCSKCSPGQHAKVFCTKTSDTVCDSCEDSTYTQLWNWVPECLSCGSRC
SSDQVETQACTREQNRICTCRPGWYCALSKQEGCRLCAPLRKCRPGFGVA
RPGTETSDVVCKPCAPGTFSNTTSSTDICRPHQICNVVAIPGNASMDAVC
TSTSPTRSMAPGAVHLPQPVSTRSQHTQPTPEPSTAPSTSFLLPMGPSPP
AEGSTGDEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYS
KLTVDKSRWQQGNVFSCSVMHEALHNHYTQKSLSLSPGKGGGGSGGGGSG
GGGSLRLLDHRAPVCSQPGLNCTVKNSTCLDDSWIHPRNLTPSSPKDLQI
QLHFAHTQQGDLFPVAHIEWTLQTDASILYLEGAELSVLQLNTNERLCVR
FEFLSKLRHHHKRWRFTFSHFVVDPGQEYEVTVHHLPKPIPDGDPNHQSK
NFLVPDCEDARMKVTTPCMSSGSLWDPNITVETLEAHQLRVSFTLWNEST
HYQILLTSFPHMENHSCFEHMHHIPAPRPEEFHQRSNVTLTLRNLKWCCR
HQVQIQPFFSSCLNDCLRHSVTVSCPEMPDTPEPIPDYMPLW

### Embodiment 2

TNFR2-Fcγ-IL-17RA-Fc Fusion Protein Expression

An encoding sequence of the fusion protein TNFR2-Fcγ-IL-17RA-Fc is cloned into a multiple cloning site of an expression vector to realize the linkage between the encoding sequence and the expression vector and to obtain plasmid DNA. The plasmid DNA transfects a host cell, and the transfection may be performed on a six-hole panel. A positive cell strain after the transfection is subjected to passage into a 1 L shake flask for shake cultivation of 120 RPM in a 5% CO₂ environment at a temperature of 37° C., nutrients such as glucose and amino acids are supplemented every day, and the cultivation is stopped when a cell viability decreases to 80-85%. After cell sap is centrifuged at 2000 RCF to take cells out, centrifugation is performed at 5000 RCF to take a supernatant for protein purification, and then the TNFR2-Fcγ-IL-17RA-Fc fusion protein is obtained.

### Embodiment 3

TNFR2-Fcγ-IL-17RA-Fc Fusion Protein Activity Experiment:

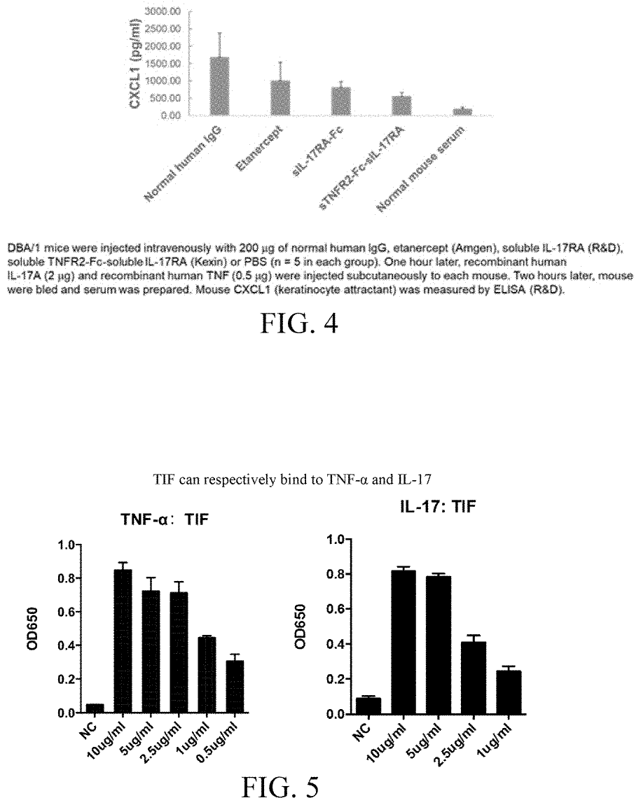

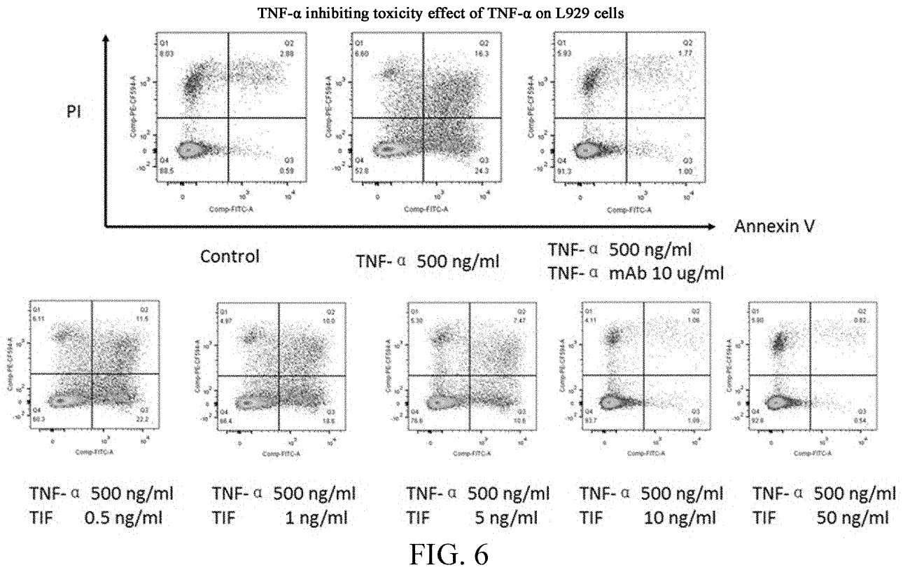

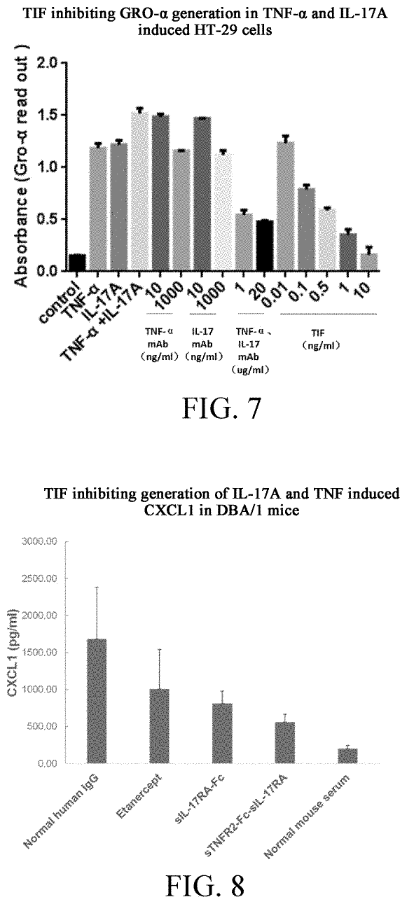

The TNF-α has a killing effect on L929 cells, so that the TNFR2-Fcγ-IL-17RA-Fc fusion protein has a protection effect on cell killing. Therefore, the bioactivity of TNFa-Fab can be detected through an antagonistic action of TNFR2-Fcγ-IL-17RA-Fc fusion protein on the killing effect of TNF-α on target cell L929 cell strains. Experiment proves that the TNFR2-Fcγ-IL-17RA-Fc fusion protein has a high protection effect on the L929 cells, indicating that the TNFR2-Fcγ-IL-17RA-Fc fusion protein has high activity and specificity for the TNF-α. See FIG. 4 to FIG. 8 for results.

Based on the above, the present invention effectively overcomes various shortcomings in the prior art and has a high industrial utilization value.

The above embodiments merely illustrate the principles and effects of the present invention, and are not intended to limit the present invention. Those skilled in the art can modify or change the above embodiments without departing from the spirit and scope of the present invention. Therefore, all equivalent modifications or changes made by a person of ordinary skill in the art without departing from the spirit and technical idea of the present invention should be covered by the claims of the present invention.

1. A bispecific fusion protein for IL-17 and TNF-α, the bispecific fusion protein being a dimer, wherein each comprises three structural function areas, and the three structural function areas are a TNF-α receptor fragment, an Fcγ fragment and an IL-17 receptor fragment;
the IL-17 receptor fragment is:
a) a polypeptide with an amino acid sequence shown as SEQ ID No. 1; or
b) a polypeptide with an amino acid sequence having 80% or more homology with the SEQ ID No. 1 and having functions of the polypeptide defined in a);
the TNF-α receptor fragment is:
c) a polypeptide with an amino acid sequence shown as SEQ ID No. 2; or
d) a polypeptide with an amino acid sequence having 80% or more homology with the SEQ ID No. 2 and having functions of a polypeptide defined in c); and
the Fcγ fragment is an Fcγ Hinge-CH2-CH3 fragment, and the Fcγ Hinge-CH2-CH3 fragment is:
e) a polypeptide with an amino acid sequence shown as SEQ ID No. 3; or
f) a polypeptide with an amino acid sequence having 80% or more homology with the SEQ ID No. 3 and having functions of a polypeptide defined in e).

2. The bispecific fusion protein for IL-17 and TNF-α according to claim 1, wherein the protein sequentially comprises the TNF-α receptor fragment, the Fcγ fragment and the IL-17 receptor fragment from an end N to an end C, and is a dimer.

3. The bispecific fusion protein for IL-17 and TNF-α according to claim 1, wherein the dimer is bonded through disulfide bonds between the Fcγ Hinge-CH2-CH3 fragments.

4. An isolated polynucleotide, encoding the bispecific fusion protein for IL-17 and TNF-α according to claim 1.

5. A recombinant expression vector, comprising a polynucleotide encoding the bispecific fusion protein for IL-17 and TNF-α according to claim 1.

6. A fusion protein expression system, comprising the recombinant expression vector according to claim 5 or the polynucleotide in which an exogenous gene is integrated into a genome.

7. A preparation method of the bispecific fusion protein for IL-17 and TNF-α according to claim 1, comprising the following steps:
1) culturing the fusion protein expression system so that the fusion protein expression system expresses the bispecific fusion protein;
2) collecting a culture material containing the bispecific fusion protein; and
3) isolating the bispecific fusion protein from the culture material obtained in step 2).

8. A composition, comprising a therapeutically effective amount of the bispecific fusion protein for IL-17 and TNF-α according to claim 1.

9. A use of the bispecific fusion protein for IL-17 and TNF-α according to claim 1 in preparation or screening of a TNFα inhibitor and/or an IL-17 inhibitor.

10. An isolated polynucleotide, encoding the bispecific fusion protein for IL-17 and TNF-α according to claim 2.

11. An isolated polynucleotide, encoding the bispecific fusion protein for IL-17 and TNF-α according to claim 3.

12. A recombinant expression vector, comprising a polynucleotide encoding the bispecific fusion protein for IL-17 and TNF-α according to claim 2.

13. A recombinant expression vector, comprising a polynucleotide encoding the bispecific fusion protein for IL-17 and TNF-α according to claim 3.

14. A preparation method of the bispecific fusion protein for IL-17 and TNF-α according to claim 2, comprising the following steps:
1) culturing the fusion protein expression system so that the fusion protein expression system expresses the bispecific fusion protein;
2) collecting a culture material containing the bispecific fusion protein; and
3) isolating the bispecific fusion protein from the culture material obtained in step 2).

15. A preparation method of the bispecific fusion protein for IL-17 and TNF-α according to claim 3, comprising the following steps:
1) culturing the fusion protein expression system so that the fusion protein expression system expresses the bispecific fusion protein;
2) collecting a culture material containing the bispecific fusion protein; and
3) isolating the bispecific fusion protein from the culture material obtained in step 2).

16. A composition, comprising a therapeutically effective amount of the bispecific fusion protein for IL-17 and TNF-α according to claim 2.

17. A composition, comprising a therapeutically effective amount of the bispecific fusion protein for IL-17 and TNF-α according to claim 3.

18. A composition, comprising a therapeutically effective amount of the bispecific fusion protein for IL-17 and TNF-α according to a culture material of the fusion protein expression system according to claim 6.

19. A use of the bispecific fusion protein for IL-17 and TNF-α according to claim 2 in preparation or screening of a TNFα inhibitor and/or an IL-17 inhibitor.

20. A use of the bispecific fusion protein for IL-17 and TNF-α according to claim 3 in preparation or screening of a TNFα inhibitor and/or an IL-17 inhibitor.


SEQUENCE LISTING (6 sequences)

SEQ ID NO: 1 - protein, 288 aa, Homo sapiens
LRLLDHRAPVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHKRWRFTFSHFVVDPGQEYEVTVHHLPKPIPDGDPNHQSKNFLVPDCEDARMKVTTPCMSSGSLWDPNITVETLEAHQLRVSFTLWNESTHYQILLTSFPHMENHSCFEHMHHIPAPRPEEFHQRSNVTLTLRNLKWCCRHQVQIQPFFSSCLNDCLRHSVTVSCPEMPDTPEPIPDYMPLW

SEQ ID NO: 2 - protein, 257 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 3 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 864 bp, Homo sapiens
ctgcgactcctggaccaccgggcgccagtctgctcccagccggggctaaactgcacggtcaagaatagtacctgcctggatgacagctggattcaccctcgaaacctgaccccctcctccccaaaggacctgcagatccagctgcactttgcccacacccaacaaggagacctgttccccgtggctcacatcgaatggacactgcagacagacgccagcatcctgtacctcgagggtgcagagttatctgtcctgcagctgaacaccaatgaacgtttgtgcgtcaggtttgagtttctgtccaaactgaggcatcaccacaaacggtggcgttttaccttcagccactttgtggttgaccctggccaggaatatgaggtgaccgttcaccacctgcccaagcccatccctgatggggacccaaaccaccagtccaagaatttccttgtgcctgactgtgaggacgccaggatgaaggtaaccacgccatgcatgagctcaggcagcctgtgggaccccaacatcaccgtggagaccctggaggcccaccagctgcgtgtgagcttcaccctgtggaacgaatctacccattaccagatcctgctgaccagttttccgcacatggagaaccacagttgctttgagcacatgcaccacatacctgcgcccagaccagaagagttccaccagcgatccaacgtcacactcactctacgcaaccttaaatggtgctgtcgccaccaagtgcagatccagcccttcttcagcagctgcctcaatgactgcctcagacactccgtgactgtttcctgcccagaaatgccagacactccagaaccaattccggactacatgcccctgtgg

SEQ ID NO: 5 - DNA, 771 bp, Homo sapiens
atggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgac

SEQ ID NO: 6 - DNA, 696 bp, Homo sapiens
gagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa